(19) United States
(12) Patent Application Publication
(10) Pub. No.: US 2007/0098672 A1
(43) Pub. Date: May 3, 2007

(54) Fogger propellant system for containing and dispersing antimicrobial and air freshening compounds

(76) Inventor: Kevin W. O'Sullivan, Las Vegas, NV (US)

Correspondence Address: Galasso & Associates, LP, P.O. Box 26503, Austin, TX, 78755-0503, US

(21) Appl. No.: 11/591,814

(22) Filed: Nov. 2, 2006

Related U.S. Application Data
(60) Provisional application No. 60/732,877, filed on Nov. 2, 2005.

(51) Int. Cl. A61L 9/00; A61M 11/00

(52) U.S. Cl. 424/76.2; 128/200.23

(57) ABSTRACT

A fogger propellant system configured to discharge an antimicrobial mixture in order to substantially inhibit growth and reproduction of microbes in the area adjacent to the system. The system further includes a container that has disposed therein a propellant and an antimicrobial compound. The antimicrobial compound is dispersed from the container in a gaseous form and further includes a fragrance oil to achieve a desirable odor during the dispersal of the antimicrobial compound. The container includes a nozzle having an aperture journaled therethrough in order to facilitate the dispersal of the antimicrobial compound. A trigger is operably connected to the nozzle to commence the release of the antimicrobial compound.

### PRIORITY UNDER 35 U.S.C. 119(e) & 37 C.F.R. 1.78

This nonprovisional application claims priority based upon the following prior U.S. provisional patent application entitled: Ultra master fogger, Application No. 60/732,877, filed Nov. 02, 2005, in the names of Kevin W. O'Sullivan and Collin O'Sullivan, which is hereby incorporated by reference for all purposes.

### FIELD OF THE INVENTION

The present invention relates to a disinfectant device, more specifically but not by way of limitation, to a system utilizing antimicrobial materials and air freshening compounds that are emitted by a fog that substantially inhibits the growth of bacteria, algae, and mold while simultaneously freshening the air.

### BACKGROUND

Millions of households are afflicted with a plurality of microbes that can cause health problems for the individuals residing in the households or be the source of unwanted mildew or molds. Houses and other structures that are located in areas of the country that have high average humidity are very susceptible to microbes that generate mold and mildew. This creates an undesirable situation for the residents or users of the structure that has been afflicted with the microbes.

One problem is that routine cleaning of the areas with commonly available household cleaning products is usually not sufficient to remove the microbes thus causing the need for significant and often expensive remediation. The chemicals used to remediate the microbes are liquid in form and can cause damage to the items to which they are applied. The microbes also create an unpleasant odor in the air within the structure that is not easily removed with household cleaning products.

Accordingly there is a need for a device that can easily treat areas such as the internal area of a room in a house and substantially penetrate the objects therein in order to inhibit the growth of microbes while not creating an unpleasant odor.

### SUMMARY OF THE INVENTION

It is the object of the present invention to provide a system that delivers to the surrounding environment an antimicrobial compound along with air freshener.

A further object of the present invention is to provide a device that emits a antimicrobial compound in a gaseous form so as to substantially distribute the compound in the area adjacent to the device.

It is an object of the present invention to provide a device that emits an antimicrobial gaseous compound that utilizes a propellant to assist in the delivery of the antimicrobial gaseous compound.

Another object of the present invention is to provide a device that emits an antimicrobial gaseous compound that is manufactured in a plurality of sizes and is relatively inexpensive.

To the accomplishment of the above and related objects the present invention may be embodied in the form illustrated in the accompanying drawings. Attention is called to the fact that the drawings are illustrative only. Variations are contemplated as being a part of the present invention, limited only the scope of the claims.

### BRIEF DESCRIPTION OF THE DRAWING

A more complete understanding of the present invention may be had by reference to the following Detailed Description and appended claims when taken in conjunction with the accompanying Drawing wherein:

FIG. 1 is a perspective view of the container that stores and subsequently emits the compounds contained therein.

### DETAILED DESCRIPTION

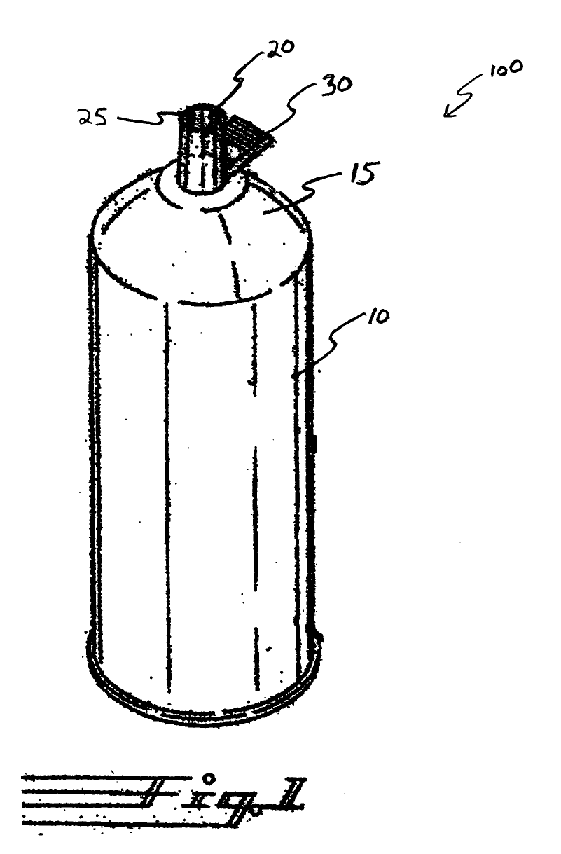

Referring now to the drawing submitted herewith wherein the various elements depicted therein are not necessarily drawn to scale and in particular FIG. 1, there is illustrated a preferred embodiment of a antimicrobial device 100 constructed according to the principles of the present invention.

The antimicrobial device 100 comprises a container 10 that is substantially cylindrical in shape and is manufactured from a durable rigid material such as aluminum or steel. The container 10 is a conventional aerosol container that is manufactured to store a variety of contents that are disposed within the container under pressure. It is contemplated within the scope of the present invention that the container 10 would have disposed therein a propellant in order to assist in the dispersal of the antimicrobial substance. Those skilled in the art will recognize that numerous different propellants could be utilized in the antimicrobial device 100. More specifically but not by way of limitation, the container 10 could utilize compressed gas, chlorofluorocarbons, LPG, or dimethyl ether as a propellant to assist in the dispersal of the antimicrobial substance.

Proximate the first end 15 of the container 10 is a nozzle 20. The nozzle 20 is generally cylindrical in shape and has an aperture 25 journaled therethrough so as to provide a path for the contents therein to be emitted from the container 10. A trigger 30 is secured to the nozzle 20 by suitable mechanical methods. The trigger 30 functions to open the aperture 25 thereby subsequently providing a path for the contents disposed within the container 10 to be released. In a preferred embodiment, container 10 contains one fluid that boils well below room temperature, the propellant, and another fluid that boils at a higher temperature, the product. The propellant is stored under high pressure and functions to propel the other compounds disposed within the container 10 out through the nozzle 20 subsequent to the user engaging the trigger 30. Although no specific size of the container 10 is required, good results have been achieved by utilizing a container 10 that is manufactured to hold approximately between eight to sixteen ounces of antimicrobial substance.

It is contemplated within the scope of the present invention that disposed within the container 10 is an antimicrobial compound that when dispersed from the container through the nozzle 20 the compound is in a gaseous form. A gaseous form of the antimicrobial substance is desired as the molecular structure of a gaseous compound is desirable for penetrating materials and covering a significant area adjacent to the antimicrobial device 100. While disposed within the container 10, the antimicrobial substance is under pressure and thus in a liquid form. Antimicrobial substances function to penetrate the cell wall of the microbe and subsequently disrupt the key cell functions of the microbe substantially inhibiting the microbe to maintain growth or reproduce. Those skilled in the art will recognize that numerous different antimicrobial substances could be utilized in the antimicrobial device 100. More specifically but not by way of limitation, the antimicrobial device 100 could utilize brominated quaternary phenolic compounds, alletherins or gluteraldehydes. It should be recognized that these compounds could be used in isolation or in any suitable combination thereof.

Disposed within the container 10 in addition to the propellants and the antimicrobial substances referenced herein are a plurality of air fresheners or deodorizers. These compounds function to provide a desired fragrance upon being released in conjunction with the antimicrobial compound. Those skilled in the art will recognize that numerous different scents of fragrance oils or perfumes could be mixed with the antimicrobial compound disposed within the container 10 to achieve a desirable fragrance during emission of the antimicrobial substance.

Referring in particular to FIG. 1, a description of the operation of the antimicrobial device 100 is as follows. In use, a user will place the container 10 in the center of the desired area that application of an antimicrobial compound is desired, generally inside a building or home. The user will agitate the container 10 and then place the container 10 in a substantially vertical position with the first end 15 being distal to the suitable support structure upon which the container 10 has been placed. The user then depresses the trigger 30 in order to release the antimicrobial compound mixture in a gaseous form. The antimicrobial mixture is dispersed and penetrates the area and objects contained therein in order to substantially inhibit microbe growth and freshen the surrounding air. Following completion of the dispersal of the antimicrobial mixture, the user disposes of the container 10 properly.

In the preceding detailed description, reference has been made to the accompanying drawing that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A fogging system, comprising:
a container, said container operable to receive and store a mixture;
a nozzle connected to said container, said nozzle operable to disperse said mixture disposed within said container; and
said mixture including at least one of an antimicrobial compound and an air freshening compound.

2. The system as recited in claim 1, and further including a propellant disposed within said container, said propellant for assisting in the dispersal of said at least one of an antimicrobial compound and an air freshening compound.

3. The system as recited in claim 2, wherein said antimicrobial compound includes a brominated quaternary phenolic compound.

4. The system as recited in claim 3, wherein said air freshening compound includes a fragrance oil.

5. The system as recited in claim 4, wherein the antimicrobial mixture is dispersable in a gaseous form from said container.

6. The system as recited in claim 5, wherein said propellant includes compressed gas, wherein the compressed gas functions to disperse said antimicrobial compound from said container.

7. A fogging propellant device, comprising:
a container, said container having a first end and a second end, said container having a sufficient volume to receive and store a mixture;
a nozzle, said nozzle connected to said container at said first end of said container, said nozzle operable to discharge said mixture; and
said mixture including at least one of an antimicrobial compound and an air freshening compound.

8. The device as recited in claim 7, and further including a propellant disposed within said container, said propellant for assisting in the dispersal of said at least one of an antimicrobial compound and an air freshening compound.

9. The device as recited in claim 8, wherein said antimicrobial compound operable to substantially inhibit the growth and reproduction of microbes.

10. The device as recited in claim 9, wherein said antimicrobial compound is dispersable from said nozzle in a gaseous form to facilitate the dispersion and penetration of said antimicrobial compound into objects adjacent to said canister.

11. The device as recited in claim 10, wherein said antimicrobial compound includes at least one of brominated quaternary phenolic compounds, alletherins and gluteraldehydes.

12. The device as recited in claim 11, wherein said air freshening compound includes a fragrance oil.

13. The device as recited in claim 12, wherein said canister stores approximately between eight to sixteen ounces of said mixture.

14. A canister system configured to emit a microbe inhibitor, comprising:
a container, said container having a first end and a second end, said container having a sufficient volume to receive and subsequently store a first and a second substance therein, said second substance having a first phase and a second phase, wherein in said first phase, said second substance is in a liquid form and is disposed within said container and in said second phase said second substance is in a gas form subsequent to release from said container; and
a nozzle, said nozzle being proximate said first end of said container, said nozzle being operable to discharge said second substance disposed from said container.

15. The canister system as recited in claim 14, wherein said second substance is an antimicrobial mixture operable to substantially inhibit growth and reproduction of microbes.

16. The canister system as recited in claim 15, wherein said second substance is manufactured from a group of compounds consisting of quaternary phenolic compounds, alletherins or gluteraldehydes.

17. The canister system as recited in claim 16, wherein said second substance further includes a fragrance oil.

18. The canister system as recited in claim 17, wherein said first substance is a propellant, said propellant being operable to discharge said second substance from said canister.

19. The canister system as recited in claim 18, wherein said first substance is a compressed gas.

20. The canister system as recited in claim 19, wherein said canister is manufacture to store approximately between eight to sixteen ounces of said second substance.
